(19) United States
(12) Patent Application Publication
(10) Pub. No.: US 2003/0207280 A1
(43) Pub. Date: Nov. 6, 2003

(54) Determination of physiological conditions defined in traditional chinese medicine

(76) Inventors: Hsiung Hsiao, Taipei (TW); Ping-Ting Huang, Kaohsiung (TW); Ching-Hsiang Hsu, Kaohsiung (TW)

Correspondence Address: FISH & RICHARDSON PC, 225 FRANKLIN ST, BOSTON, MA, 02110, US

(21) Appl. No.: 10/139,711

(22) Filed: May 6, 2002

(51) Int. Cl. C12Q 1/68; G06F 19/00; G01N 33/48; G01N 33/50

(52) U.S. Cl. 435/6; 702/20

(57) ABSTRACT

A substrate containing detection agents for determining expression levels of a plurality of genes. A combination of weighted expression levels of the genes is indicative of a physiological condition defined in traditional Chinese medicine. Also disclosed are software and a method for correlating gene expression levels to a physiological condition defined in traditional Chinese medicine.

### BACKGROUND

Traditional Chinese medicine has been in existence for several thousands of years and is based largely on accumulated experience in fighting various diseases through the long history of Chinese civilization. It deals with pathology, and diagnosis, treatment and prevention of diseases. Although traditional Chinese medicine has been proved to be effective in many cases, its underlying principles have largely not been interpreted in scientific terms.

### SUMMARY

The present invention relates to determining a physiological condition defined in traditional Chinese medicine by measuring expression levels of a plurality of genes.

In one aspect, this invention features a substrate containing detection agents for determining expression levels (e.g., mRNA levels or protein levels) of a plurality of genes. A combination of weighted expression levels of the genes is indicative of a physiological condition defined in traditional Chinese medicine (e.g., Yin [symbol] /Yan [symbol] , Cold [symbol] /Heat [symbol] , Deficiency [symbol] /Excessiveness [symbol] , and Exterior [symbol] /Interior [symbol] ). The detection agents can be nucleic acids (e.g., primers and probes) for measuring mRNA levels, or peptides (e.g., binding proteins, antibodies and ligands) for measuring protein levels. The gene expression levels can be weighted and combined according to any given mathematical formula (e.g., aX₁+bX₂, aX₁−bX₂, aX₁×bX₂, aX₁/bX₂, and aX₁^{m}+bX₂ⁿ; X₁ and X₂ representing the expression levels of two selected genes; a, b, m, and n are constants). The substrate of the invention can be used to determine a physiological condition defined in traditional Chinese medicine.

In another aspect, this invention features software configured to instruct a processor to receive information including a score for a physiological condition defined in traditional Chinese medicine and expression levels of a plurality of genes (e.g., determined as described above), and derive a formula to correlate the score to a combination of weighted expression levels of the genes (e.g., by multiple regression analysis). The software of the invention can be used to determine which and how gene expression levels are related to a physiological condition defined in traditional Chinese medicine.

In yet another aspect, this invention features software configured to instruct a processor to receive information including expression levels of a plurality of genes and determine a combination of weighted expression levels of the genes according to a formula derived as described above. The software of the invention is used to determine a physiological condition defined in traditional Chinese medicine according to the expression levels of a certain set of genes.

Also within the scope of this invention is a method of determining a physiological condition defined in traditional Chinese medicine. The method includes quantifying expression levels of a plurality of genes, and combining them such that the combination of weighted expression levels of the genes is indicative of a physiological condition defined in traditional Chinese medicine. Quantification of gene expression levels and determination of a relevant physiological condition defined in traditional Chinese medicine can be performed as described above.

The present invention provides a novel method which enables one to determine a physiological condition defined in traditional Chinese medicine without performing diagnostic procedures used in traditional Chinese medicine and facilitates evaluation of the efficacy of traditional Chinese medicine and treatment of patients with traditional Chinese medicine. The details of one or more embodiments of the invention are set forth in the accompanying description below. Other features, objects, and advantages of the invention will be apparent from the detailed description, and from the claims.

### DETAILED DESCRIPTION

The “8 Principles” (or “8 Conditions,” or “Ba [symbol] Gang [symbol] ” in Chinese) is used to classify physiological conditions of a human body for diagnosis and treatment in traditional Chinese medicine (TCM). The basic physiological conditions of the “8 Principles” are Yin/Yan, Cold/Heat, Deficiency/Excessiveness, and Exterior/Interior. Among them, the most commonly used are Cold/Heat and Deficiency/Excessiveness. The basic physiological conditions can be further categorized into sub-physiological conditions according to the type of a disease and the type of an organ involved.

According to TCM, diseases are caused by imbalance of one or more of the physiological conditions. Thus, accurately identifying an imbalanced physiological condition is critical for treatment of the disease. In TCM, a physiological condition is determined by visual inspection, auscultation, questioning, and palpation (Si [symbol] Zhen [symbol] : [symbol] , [symbol] , [symbol] , [symbol] in Chinese). Visual inspection involves an evaluation of general appearance and complexion, attitudes and movements, and facial expression. It can also include examination of a patient's excreta. Auscultation involves listening to the sounds (e.g., voice and breathing) of a patient. Questioning involves recording the full ananmesis of a patient, including the health history, the present disease, and the present symptoms. Palpation (including pulse diagnosis) is carried out by light touch or deep pressure with fingertips.

The present invention is based on the discovery that physiological conditions defined in traditional Chinese medicine correlate with gene expression levels. It provides a method of identifying a physiological condition defined in traditional Chinese medicine without performing the diagnostic procedures described above.

Specifically, the method of the invention includes quantifying expression levels of a plurality of genes, and obtaining a score by combining weighted gene expression levels according to a mathematical formula. The score indicates the state of a physiological condition defined in traditional Chinese medicine, e.g., the degree of Cold/Heat.

A group of genes are initially chosen according to their alleged relevance to the physiological condition to be determined. For instance, genes highly related to immune and inflammatory responses can be chosen for determining a physiological condition associated with asthma (see the example below). The relevance of a gene can be verified according to the method of this invention (described below). Some of the genes may be found not to contribute to the physiological condition. These genes can then be taken out from the group. Additional genes thought to be relevant to the physiological condition can be included in the gene group for further verification.

The expression of each gene can be quantified at the mRNA level or at the protein level. Methods for quantifying a specific mRNA or protein are well known in the art. Typically, a sample to be analyzed is prepared from a biopsy (e.g., a tissue or cell sample) or a body fluid (e.g., a blood or urine sample). One or more detection agents can be used for each gene. The detection agents for each gene can be either separately localized to a compartment of a substrate (e.g., a membrane, microcentrifuge tube, microtiter plate, and silicon or glass slide) or mixed together. They can be either attached to the substrate or in a free state (e.g., in a solution). The mRNA level can be measured using a number of techniques including Southern or Northern blotting, the polymerase chain reaction, and microarray analysis. The detecting agents include nucleic acid primers and probes. See, e.g., Schena et al. (1995) Science 270:467470; Eisen and Brown (1999) Methods Enzymol. 303:179-205; Blohm and Guiseppi-Elie (2001) Curr. Opinion in Biotechnol. 12:41-47; Mullis (1987) U.S. Pat. No. 4,683,202; Barany (1991) Proc. Natl. Acad. Sci. USA 88:189-193; Guatelli et al. (1990) Proc. Natl. Acad. Sci. USA 87:1874-1878; Kwoh et al. (1989) Proc. Natl. Acad. Sci. USA 86:1173-1177; and Lizardi et al. (1988) BioTechnology 6:1197. A variety of methods can be used to determine the level of a specific protein. In general, these methods include contacting a sample with a detecting agent that selectively binds to a target protein (e.g., an antibody or a ligand) to evaluate the level of the protein in the sample. The commonly used methods for detecting a specific protein include enzyme linked immunosorbent assay (ELISA), immunoprecipitation, immunofluorescence, enzyme immunoassay (EIA), radioimmunoassay (RIA), Western bloting, protein chip analysis. See, e.g., Harlow and Lane (1988) Antibodies. A laboratory manual. Cold Spring Harbor Laboratory; Celis et al. (1994) Determination of antibody specificity by Western blotting and immunoprecipitation. In Cell Biology. A Laboratory Handbook. Celis, J. E. (ed.), Academic Press, New York, Vol. 2, pp. 305-313; Porstmann and Kiessig (1992) J. Immunol. Methods 150:5-21; Dwenger (1984) J. Clin. Biochem. 22:883; and MacBeath and Schreiber (2000) Science 289:1760.

In order to identify which and how gene expression levels correlate with a physiological condition defined in traditional Chinese medicine, a set of human subjects are subject to both examination by a TCM practitioner using the diagnostic procedures described above and gene expression analysis at the same time. For each human subject, a score for a physiological condition is assigned by the TCM practitioner, and the gene expression levels are determined using the methods described above. The relationship between the scores and the gene expression levels is then evaluated, e.g., using multiple regression analysis software. A mathematical formula is derived, including only the gene expression levels found to be statistically related to the physiological condition. See the example below. The formula can be modified for improved accuracy by including more genes and more human subjects in the evaluation process. Once a formula is established, it can be used to determine the state of a physiological condition by measuring the expression levels of relevant genes and obtaining a score according to the formula. As shown in the example below, a formula can be established to represent the correlation between the expression levels of 31 genes and one of the physiological conditions defined in traditional Chinese medicine, “Cold/Heat,” for asthma patients. Using this formula, a patient's “Cold/Heat” condition can be predicted with an accuracy of approximate 87%.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Asthma patients of both genders and different ages were examined at the same time by one TCM practitioner and one medical professional or physician during their visits. “Cold/Heat” and “Deficiency/Excessiveness” conditions of each patient were recorded and scored by the TCM practitioner as follows:

TABLE 1
Cold/Heat Condition Scoring System for Asthma Patients

| Symptoms | Severity | Score |
|---|---|---|
| Feel thirsty and like cold drinks | Frequently | 2 |
| | Occasionally | 1 |
| | None | 0 |
| Constipation | Frequent | 2 |
| | Occasional | 1 |
| | None | 0 |
| Yellow urine | Frequent | 2 |
| | Occasional | 1 |
| | None | 0 |
| Red facial complexion | Obvious | 2 |
| | Mild | 1 |
| | None | 0 |
| Red lips | Obvious | 2 |
| | Mild | 1 |
| | None | 0 |
| Red eyes | Obvious | 2 |
| | Mild | 1 |
| | None | 0 |
| Red tongue | Obvious | 2 |
| | Mild | 1 |
| | None | 0 |
| Tongue fur | Yellow grimy | 2 |
| | Thin yellow | 1 |
| | Not yellow | 0 |

TABLE 2
Deficiency/Excessiveness Condition Scoring System for Asthma Patients

| Symptoms | Severity | Score |
|---|---|---|
| Fatigued spirit and lack of | Frequent | 2 |
| strength | Occasional | 1 |
| | None | 0 |
| Don't feel like to speak and | Frequently | 2 |
| like sleeping | Occasionally | 1 |
| | None | 0 |
| 1. Spontaneous sweating | Frequent | 2 |
| 2. Easy sweating | Occasional | 1 |
| | None | 0 |
| Bright, white facial complexion | Frequent | 2 |
| | Occasional | 1 |
| | None | 0 |
| 1. Enduring cough | Frequent | 2 |
| 2. Persisting cough and lack of | Occasional | 1 |
| strength | None | 0 |
| Whitish sputum | Frequent | 2 |
| | Occasional | 1 |
| | None | 0 |
| Dental impressions on the | Yes | 1 |
| margin of the tongue | None | 0 |
| Pulse | Strong | 1 |
| | Weak | 0 |

Also, for each patient, three to five milliliters of whole blood were collected and processed as follows:

Processing Clinical Samples

1. Collect 3˜5 ml whole blood sample in EDTA.

2. Isolate white blood cells using Ficoll-Paque® (Pharmacia Biotech):

(1) Add 5 ml Ficoll to a 15 ml centrifuge tube.

(2) Add blood to the tube.

(3) Centrifuge at 25,000 rpm for 20 min.

(4) Transfer the serum (yellow color, the first layer) to a 1.5 ml microcentrifuge tube and store at −20° C.; transfer the buffy coat (white and cloudy, the second layer) to a 15 ml tube.

(5) Wash the buffy coat with 1×PBS, centrifuge at 15,000 rpm for 10 min.

(6) Discard the liquid, resuspend the pellet in 1×PBS, mix well, and centrifuge at 15,000 rpm for 10 min.

(7) Discard the liquid, and resuspend the pellet in 300 μl 1×PBS.

(8) Add 5×vol. of RNA Later (1500 μl), mix by gently inverting the capped tube 5˜6 times.

(9) Store at −20° C.

Extraction of Total RNA

1. Spin down the lymphocytes (in RNA Later) at 400×g for 10 minutes at 4° C.

2. Add 1 ml TRIZOL reagent (Life Technologies) and vortex vigorously to lyse the cells.

3. Incubate on ice for 5 minutes and then spin briefly.

4. Add 0.2 ml CHCl₃ and vortex vigorously for 1 minute.

5. Incubate on ice for 2 minutes.

6. Centrifuge at 14,000×g for 15 minutes at 4° C.

7. Transfer the supernatant (about 0.6 ml) to a new 1.5 ml tube.

8. Add 0.5 ml isopropanol and mix well.

9. Incubate at −20° C. for 20 minutes.

10. Centrifuge at 14,000×g for 15 minutes at 4° C.

11. Discard the supernatant.

12. Rinse the RNA pellet with 75% ethanol.

13. Centrifuge at 14,000×g for 5 minutes at 4° C.

14. Discard the supernatant and vacuum dry the pellet.

15. Resuspend the pellet in 20 μL RNase-free water.

16. Determine the concentration of total RNA using spectrophotometer.

Cy5 Labeling and Purification

1. Prepare sufficient 2× reverse transcription labeling mixture and store the solution at −20° C. 2× reverse transcription labeling mixture contains:

| | |
|---|---|
| 5× reverse transcription buffer | 120 μL |
| DTT (5 mM) | 60 μL |
| dATP (100 mM) | 3 μL |
| dCTP (100 mM) | 3 μL |
| dGTP (100 mM) | 3 μL |
| dTTP (100 mM) | 0.6 μL |
| Nuclease-free water | 110.4 μL |
| Total volumn | 300.0 μL |

2. Mix 2 μL, 0.1 μg/μL oligo-dT (12-18-mer; Life Technologies) with 8 μL total RNA (If the initial RNA concentration is greater than 0.3 μg/μL, dilute the solution to about 0.3 μg/μL; if the RNA concentration is between 0.15-0.3 μg/μL, dilute the solution to about 1.5 μg/μL).

3. Incubate the mixture at 70° C. for 10 minutes and snap cool on ice for 2 to 3 minutes.

4. Add the following reagents to the mixture in a dark room and mix thoroughly:

| | |
|---|---|
| 2× reverse transcription labeling mixture | 16 μL |
| Cy5-dUTP (1 mM) | 3 μL |
| SuperScript II (200 U/μL) | 2 μL |
| RNAsin | 1 μL |

5. Incubate the mixture at 42° C. for 2 hours.

6. Add 1.5 μL, 20 mM EDTA to stop the reverse transcription.

7. Add 1.5 μL, 500 mM NaOH and heat at 72° C. for 10 minutes to degrade the RNA.

8. Add 1.5 μL, 500 mM HCl to neutralize the mixture.

9. Remove unincorporated fluorescent nucleotides using ProbeQuant G-50 Micro Column (Amersham Pharmnacia Biotech).

10. Dry the purified mixture in a vacuum dryer and resuspend the pellet in 10 μL human COT-1 DNA and 1 μL, 20 μg/μL poly-A RNA.

11. Store the Cy5-labeled sample at −80° C.

Preparation of Microarray Slides

Microarray slides were prepared following standard protocols. For cDNA probes, 93 probes derived from 69 genes highly related to immune and inflammatory responses were chosen for this example (see Table 3 below). For some of the genes (e.g., ACHE), multiple probes derived from different regions of the gene were used. Data thus obtained were analyzed separately (see below). In addition, a cDNA probe for GAPDH, a housekeeping gene whose expression level remains constant, was selected as a positive control. The expression levels of other genes were calibrated against the GAPDH expression level. Probes for RbCL and ATBS, two plant genes, served as negative controls.

The cDNA clones were purchased from Incyte Genomics Inc. (St. Louis, Mo., USA).

Probes were arranged in an 8×12 array pattern on slides and were spotted in duplicates.

TABLE 3
Array Pattern of cDNA Gene Probes

| | 1 | 2 | 3 | 4 | 5 | 6 | 7 | 8 | 9 | 10 | 11 | 12 |
|---|---|---|---|---|---|---|---|---|---|---|---|---|
| 1 | ACHE | CCR1 | CD31 | Colony | GBP1 | IL12 | interleu- | IRF4 | Metallo- | MUC2 | SCYA4 | STAT6 |
| | | | | stimulat- | | receptor, | kin 18 | | thionein | | | |
| | | | | ing factor | | beta 2 | receptor | | | | | |
| 2 | ACHE | CCR3 | CD34 | CXCR3 | GBP2 | IL12 | interleu- | IRF4 | Metallo- | MUC5AC | Selectin L | TBXA2R |
| | | | | (GPR9) | | receptor, | kin 4 | | thionein | | | |
| | | | | | | beta 2 | | | | | | |
| 3 | Adenylate | CCR5 | CD38 | EGR2 | HOXA1 | interleu- | IL 4 | ITGA6 | Metallo- | PDE4B | SLAM | TBXA2R |
| | Cyclase 1 | | | | | kin 13 | receptor, | | thionein | | | |
| | | | | | | | alpha | | | | | |
| 4 | Adenylate | CCR7 | CD69 | eotaxin | HOXA1 | interleu- | IL 5 | ITGA7 | MIG | PDPK | STAT1 | TBXA2R |
| | Cyclase 1 | | | | | kin 15 | receptor, | | | | | |
| | | | | | | | alpha | | | | | |
| 5 | Adenylate | CD2 | CD97 | EST1 | ICAM1 | interleu- | IL 5 | LAMR1 | MUC1 | PRKG1 | STAT2 | terminal |
| | Cyclase 1 | | | | | kin 15 | receptor, | | | | | transfer- |
| | | | | | | | alpha | | | | | ase |
| 6 | ADRB2 | CD26 | CDH3 | EST1 | ICAM2 | interleu- | IL 5 | lympho- | MUC2 | PTGER2 | STAT4 | GAPDH |
| | | | | | | kin 15 | receptor, | tacin beta | | | | |
| | | | | | | | alpha | | | | | |
| 7 | aldehyde | CD30 | CEBPB | GATA1 | interferon | interleu- | interleu- | MCP-3 | MUC2 | PANTES | STAT4 | RbCL |
| | dehydro- | | | | 1 | kin 15 | kin 6 | | | | | |
| | genase | | | | | | | | | | | |
| 8 | ANXA3 | CD30 | c-fos | GATA3 | interleu- | interleu- | IRF4 | Metallo- | MUC2 | SCYA17 | STAT4 | ATBS |
| | | | | | kin 10 | kin 18 | | thionein | | | | |

Pre-Hybridization of Micro Array Slides

1. Incubate slides in 5×SSC, 0.1% SDS, and 1% BSA in ajar for 45 minutes at 42° C.

2. Rinse slides with double distilled water.

3. Blow dry slides with compressed air.

Preparation of Hybridization Samples (Cy5-Labeled cDNA)

1. Add 11 μL, 2× hybridization buffer (50% formamide, 10×SSC, 0.2% SDS) to each sample.

2. Heat the sample at 95° C. for 3 minutes.

3. Spin 2 minutes to cool down the sample.

Hybridization

1. Preheat the hybridization oven to 42° C.

2. Clean cover slips with ddH₂O followed by 100% EtOH.

3. Dry cover slips with compressed air.

4. Add samples to the spotting area, and carefully place a cover slip on top of the slide such that no air bubbles appear under the cover slip.

5. Add 5 μl, 5×SSC in the hybridization chamber to ensure a constant humidity in the chamber during hybridization.

6. Assemble the hybridization chamber and carefully place the chamber in the hybridization oven, incubate at 42° C., overnight.

Post-Hybridization Wash

1. Disassemble the hybridization chamber and remove the slide.

2. Place slide in a slide rack, then submerge the rack in wash solution (1) (1×SSC, 0.1% SDS) at 42° C. Shake the rack gently, the coverslip shall fall off automatically.

3. Transfer the slide to a new rack and submerge the rack in wash solution (1) (1×SSC, 0.1% SDS) for 5 minutes at 42° C. with gentle shake.

4. Transfer the slide to wash solution (2) (0.1×SSC, 0.1% SDS) for 12 minutes at room temperature with gentle shake.

5. Transfer the slide to wash solution (3) (0.1×SSC) for 1 minute at room temperature with gentle shake. Repeat 4 times.

6. Rinse with running ddH₂O for 5 sec.

7. Rinse with 100% EtOH.

8. Dry the slides with compressed air.

9. Scan the slide.

Determination of Gene Expression Levels

After hybridization, microarray slides were scanned with GenePix 4000B Reader (Axon Instruments, Inc.) to measure the signal intensity of each spot. The signal intensity was then transformed into an intensity value by using the GenePix Pro 3.0 (Axon Instruments, Inc.) software. The intensity of each gene expression signal was calibrated against the intensity of GAPDH expression signal to obtain a Gene Expression Index that represent of the expression level of the gene:

Gene Expression Index=(Gene expression intensity−background intensity)÷(GAPDH expression intensity−background intensity)

Statistic Analysis of Gene Expression Data

Statistica software (Statsoft Company, OK, USA) was used to analyze the gene expression data and its correlation with the Cold/Heat and Deficiency/Excessiveness scores.

TABLE 4
Correlation between Gene Expression Levels and Cold/Heat Condition

| Gene | p-value | Gene | p-value | Gene | p-value |
|---|---|---|---|---|---|
| ACHE_1 | 0.554 | SLAM | 0.234 | MUC2_2 | 0.161 |
| CCR1 | 0.280 | TBXA2R_2 | 0.976 | PTGER2 | 0.546 |
| CD_31 | 0.571 | Adenylate Cyclase 1_2 | 0.247 | STAT4_1 | 0.065 |
| Colony stimulating | 0.864 | CCR7 | 0.210 | aldehyde | 0.531 |
| factor | | | | dehydrogenase | |
| GBP1 | 0.090 | CD_69 | 0.768 | CD_30_1 | 0.297 |
| IL12 receptor, beta 2_1 | 0.186 | EOTAXIN | 0.384 | CEBPB | 0.904 |
| interleukin 18 receptor | 0.515 | HOXA1_2 | 0.214 | GATA1 | 0.552 |
| IRF4 | 0.285 | interleukin 15_1 | 0.869 | interferon 1 | 0.063 |
| Metallothionein_1 | 0.315 | IL 5 receptor, alpha_1 | 0.218 | interleukin 15_4 | 0.525 |
| MUC2_1 | 0.304 | ITGB7 | 0.140 | interleukin 6 | 0.916 |
| SCYA4 | 0.314 | MIG | 0.823 | MCP_3 | 0.916 |
| STAT6 | 0.295 | PDPK | 1.000 | MUC2_3 | 0.924 |
| ACHE_2 | 0.421 | STAT1 | 0.010 | RANTES | 0.577 |
| CCR3 | 0.720 | TBXA2R_3 | 0.221 | STAT4_2 | 0.067 |
| CD_34 | 0.705 | Adenylate Cyclase 1_3 | 0.490 | ANXA3 | 0.401 |
| CXCR3 | 0.177 | CD_2 | 0.232 | CD_30_2 | 0.213 |
| GBP2 | 0.913 | CD_97 | 0.439 | C_FOS | 0.734 |
| IL12 receptor, beta 2_2 | 0.409 | ETS1_1 | 0.800 | GATA3 | 0.432 |
| interleukin 4 | 0.383 | ICAM1 | 0.098 | interleukin 10 | 0.791 |
| IRF4 | 0.384 | interleukin 15_2 | 0.930 | interleukin 18 | 0.575 |
| Metallothionein_2 | 0.921 | IL 5 receptor, alpha_2 | 0.906 | IRF4_2 | 0.207 |
| MUC5AC | 0.393 | LAMR1 | 0.062 | Metallothionein_4 | 0.620 |
| SELECTIN L | 0.055 | MUC1 | 0.783 | MUC2_4 | 0.195 |
| TBXA2R_1 | 0.112 | PRKG1 | 0.260 | SCYA17 | 0.762 |
| Adenylate Cyclase 1_1 | 0.989 | STAT2 | 0.032 | STAT4_3 | 0.438 |
| CCR5 | 0.183 | terminal transferase | 0.455 | | |
| CD_38 | 0.673 | ADRB2 | 0.600 | | |
| EGR2 | 0.159 | CD_26 | 0.410 | | |
| HOXA1_1 | 0.557 | CDH3 | 0.176 | | |
| interleukin 13 | 0.019 | ETS1_2 | 0.316 | | |
| IL 4 receptor, alpha | 0.915 | ICAM2 | 0.220 | | |
| ITGA6 | 0.115 | interleukin 15_3 | 0.177 | | |
| Metallothionein_3 | 0.123 | IL 5 receptor, alpha_3 | 0.751 | | |
| PDE4B | 0.822 | lymphotacin beta | 0.797 | | |

TABLE 5
Correlation between Gene Expression Levels and Deficiency/Excessiveness Condition

| Gene | p-value | Gene | p-value | Gene | p-value |
|---|---|---|---|---|---|
| ACHE_1 | 0.862 | SLAM | 0.347 | MUC2_2 | 0.981 |
| CCR1 | 0.613 | TBXA2R_2 | 0.730 | PTGER2 | 0.558 |
| CD_31 | 0.665 | Adenylate Cyclase 1_2 | 0.713 | STAT4_1 | 0.803 |
| Colony stimulating | 0.463 | CCR7 | 0.077 | aldehyde | 0.437 |
| factor | | | | dehydrogenase | |
| GBP1 | 0.898 | CD_69 | 0.303 | CD_30_1 | 0.405 |
| IL12 receptor, beta 2_1 | 0.559 | EOTAXIN | 0.686 | CEBPB | 0.348 |
| interleukin 18 receptor | 0.750 | HOXA1_2 | 0.290 | GATA1 | 0.820 |
| IRF4 | 0.459 | interleukin 15_1 | 0.693 | interferon 1 | 0.891 |
| Metallothionein_1 | 0.942 | IL 5 receptor, alpha_1 | 0.351 | interleukin 15_4 | 0.693 |
| MUC2_1 | 0.259 | ITGB7 | 0.438 | interleukin 6 | 0.838 |
| SCYA4 | 0.283 | MIG | 0.151 | MCP_3 | 0.696 |
| STAT6 | 0.273 | PDPK | 0.913 | MUC2_3 | 0.456 |
| ACHE_2 | 0.386 | STAT1 | 0.894 | RANTES | 0.115 |
| CCR3 | 0.832 | TBXA2R_3 | 0.774 | STAT4_2 | 0.872 |
| CD_34 | 0.593 | Adenylate Cyclase 1_3 | 0.637 | ANXA3 | 0.832 |
| CXCR3 | 0.881 | CD_2 | 0.229 | CD_30_2 | 0.713 |
| GBP2 | 0.439 | CD_97 | 0.644 | C_FOS | 0.949 |
| IL12 receptor, beta 2_2 | 0.312 | ETS1_1 | 0.328 | GATA3 | 0.548 |
| interleukin 4 | 0.453 | ICAM1 | 0.919 | interleukin 10 | 0.493 |
| IRF4 | 0.787 | interleukin 15_2 | 0.988 | interleukin 18 | 0.860 |
| Metallothionein_2 | 0.870 | IL 5 receptor, alpha_2 | 0.448 | IRF4_2 | 0.923 |
| MUC5AC | 0.671 | LAMR1 | 0.159 | Metallothionein_4 | 0.983 |
| SELECTIN L | 0.431 | MUC1 | 0.587 | MUC2_4 | 0.491 |
| TBXA2R_1 | 0.980 | PRKG1 | 0.107 | SCYA17 | 0.132 |
| Adenylate Cyclase 1_1 | 0.763 | STAT2 | 0.933 | STAT4_3 | 0.326 |
| CCR5 | 0.950 | terminal transferase | 0.149 | | |
| CD_38 | 0.823 | ADRB2 | 0.627 | | |
| EGR2 | 0.758 | CD_26 | 0.112 | | |
| HOXA1_1 | 0.550 | CDH3 | 0.717 | | |
| interleukin 13 | 0.553 | ETS1_2 | 0.334 | | |
| IL 4 receptor, alpha | 0.461 | ICAM2 | 0.578 | | |
| ITGA6 | 0.729 | interleukin 15_3 | 0.351 | | |
| Metallothionein_3 | 0.792 | IL 5 receptor, alpha_3 | 0.825 | | |
| PDE4B | 0.775 | lymphotacin beta | 0.861 | | |

If 0.01<p<0.05, the results are significant.

If 0.001<p<0.01, the results are highly significant.

If p<0.001, the results are very highly significant.

If p>0.05, the results are considered not statistically significant.

If 0.05<p<0.1, a trend toward statistical significance is sometimes noted.

The Forward Stepwise function of Statitica was applied to perform a multiple regression analysis in order to formulize the correlations between the gene expressions index numbers (independent variables) and the Cold/Heat and Deficiency/Excessiveness scores (dependent variables). Specifically, the software performed a partial F-test of the 93 independent variables and picked the one with the highest F value (i.e., the one with the most significant correlation with dependent variables) as the first independent variable. The software then performed the calculation with the first independent variable included in the formula to find the second independent variable with the highest F value. Again, the second independent variable was subsequently included in the formula and the software performed a further calculation. The cycle continued until the F value fell below 1.4 and the overall F value was above the standard value.

Unexpectedly, a formula was established to represent the correlation between the gene expression index numbers and the Cold/Heat score (see below). Using this formula, a patient's Cold/Heat condition was predicted with an accuracy of approximate 87%.

Cold/Heat score=9.266−4.297X₁+14.195X₂−63.813X₃−14.625X₄+0.669X₅−

18.968X₆+35.786X₇−28.364X₈−0.622X₉−7.628X₁₀+16.972X₁₁−3.161X₁₂+20.004X₁₃−5.297X₁₄+4.794X₁₅−41.230X₁₆+27.656X₁₇+0.587X₁₈−0.353X₁₉−5.617X₂₀+0.965X₂₁+26.581X₂₂−34.130X₂₃+13.134X₂₄−15.014X₂₅−13.268X₂₆+34.639X₂₇−45.892X₂₈+57.490X₂₉+12.426X₃₀+1.104X₃₁

X₁-X₃1 are gene expression index numbers for STAT1, TBXA2R_{—}1, CDH3, Interleukin 5 Receptor_{—}1, Interleukin 15_{—}3, PRKG1, Metallothionein_{—}1, STAT4_{—}1, Interleukin 13, ICAM1, Adenylate Cyclasel_{—}3, Interleukin 6, STAT2, Interferon 1, GBP1, C_FOS, Colony stimulating, Metallothionein_{—}3, TBXA2R_{—}2, Interleukin 15_{—}1, Interleukin 4, Interleukin 15_{—}4, Interleukin 18 Receptor, ETS1_{—}1, ETS1_{—}2, TBXA2R_{—}3, CCR3, SLAM, MIG, ADRB2, and Selectin L, respectively.

p value=7.48E-10

R²=0.874

Total SS=616.72, Reg SS=471.00, Res SS=145.72

Overall F=6.26

Similarly, a formula was established to represent the correlation between the gene expression index numbers and the Deficiency/Excessiveness score as follows:

Deficiency/Excessiveness score=2.707−2.685X₃₂+0.948X₃₃+10.660X₃₄−6.126X₂₅−

0.159X₃₅−56.577X₃₆+29.078X₃₇+10.106X₃₈−52.797X₃₉−72.409X₄₀+16.141X₄₁−33.123X₄₂−11.597X₄₃+0.283X₄₄+30.648X₄₅+1.511X₄₆−25.911X₆+6.110X₅+9.539X₁₀−4.320X₁₄+48.717X₄₇−38X₄₈+31.497X₄₉+1.553X₅₀+0.936X₅₁−28.684X₂₆+30.478X₂−62.133X₅₂+53.074X₂₉+24.880X₃−6.084X₁₂+5.534X₅₃−0.697X₂₁+30.439X₅₄−10.031X₅₅

X₃₂-X₅₅ are gene expression index numbers for CCR7, LAMR1, CD_{—}31, CD_{—}2, Terminal transferase, HOXA1_{—}2, ITGA6, MUC1, CD_{—}26, Adenylate cyclasel_{—}1, Interleukin 18, STAT6, CD_{—}69, MUC24, GBP2, CCR5, ACHE_{—}1, Adenylate cyclase1_{—}2, SCYA17, CXCR3, MUC2_{—}1, PDPK, Interleukin 12 Receptor beta2_{—}2, and Metallothionein_{—}2, respectively.

p value=3.47E-08

R²=0.873

Total SS=453.68, Reg SS=345.41, Res SS=108.27

Overall F=5.10

p-value represents how significant the results are without performing repeated significance tests at different a levels. p<0.005 indicates that the results are very highly significant.

R² represents the proportion of the variance of a score that can be explained by the variable x, and all the data points fall on the regression line.

The total sum of squares (total SS) is the sum of squares of the deviations of the individual sample points from the sample mean. The regression sum of squares (Reg SS) is the sum of squares of the regression components. The residual sum of squares (Res SS) is the sum of squares of the residual components. The criterion for goodness of fit is the ratio of the regression sum of squares to the residual sum of squares. A large ratio (e.g., >0.7) indicates a good fit, whereas a small ratio indicates a poor fit.

Overall F represents the F-value of the formula.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A substrate comprising detection agents for determining expression levels of a plurality of genes, wherein a combination of weighted expression levels of the genes is indicative of a physiological condition defined in traditional Chinese medicine.

2. The substrate of claim 1, wherein the detection agents are nucleic acids.

3. The substrate of claim 1, wherein the detection agents are peptides.

4. The substrate of claim 3, wherein the peptides are antibodies.

5. The substrate of claim 3, wherein the peptides are ligands.

6. The substrate of claim 1, wherein the physiological condition is “Cold/Heat.”

7. The substrate of claim 1, wherein the physiological condition is “Deficiency/Excessiveness.”

8. The substrate of claim 1, wherein the combination of weighted expression levels is a sum of weighted expression levels.

9. The substrate of claim 8, wherein the physiological condition is “Cold/Heat.”

10. The substrate of claim 8, wherein the physiological condition is “Deficiency/Excessiveness.”

11. Software configured to instruct a processor to perform steps comprising receiving information including expression levels of a plurality of genes, and determining a combination of weighted expression levels of the genes, wherein the combination of weighted expression levels of the genes is indicative of a physiological condition defined in traditional Chinese medicine.

12. Software configured to instruct a processor to perform steps comprising receiving information including a score for a physiological condition defined in traditional Chinese medicine and expression levels of a plurality of genes, and deriving a formula to correlate the score to a combination of weighted expression levels of the genes.

13. The software of claim 12, wherein the formula is derived by multiple regression analysis.

14. A method of determining a physiological condition defined in traditional Chinese medicine, the method comprising quantifying expression levels of a plurality of genes, wherein a combination of weighted expression levels of the genes is indicative of a physiological condition defined in traditional Chinese medicine.

15. The method of claim 14, wherein the gene expression levels are quantified with nucleic acids.

16. The method of claim 14, wherein the gene expression levels are quantified with peptides.

17. The method of claim 16, wherein the peptides are antibodies.

18. The method of claim 16, wherein the peptides are ligands.

19. The method of claim 14, wherein the physiological condition is “Cold/Heat.”

20. The method of claim 14, wherein the physiological condition is “Deficiency/Excessiveness.”

21. The method of claim 14, wherein the combination of weighted expression levels is a sum of weighted expression levels.

22. The method of claim 21, wherein the physiological condition is “Cold/Heat.”

23. The method of claim 21, wherein the physiological condition is “Deficiency/Excessiveness.”
